(19) United States
(12) Patent Application Publication
(10) Pub. No.: US 2021/0308166 A1
(43) Pub. Date: Oct. 7, 2021

(54) ANTIOXIDANT COMPOSITION COMPRISING NOVEL GINSENOSIDE

(71) Applicant: AMOREPACIFIC CORPORATION, Seoul (KR)

(72) Inventors: Yong Deog HONG, Yongin-si, Gyeonggi-do (KR); Hyun Woo JEONG, Yongin-si, Gyeonggi-do (KR)

(73) Assignee: AMOREPACIFIC CORPORATION, Seoul (KR)

(21) Appl. No.: 17/266,567

(22) PCT Filed: Jul. 2, 2019
(86) PCT No.: PCT/KR2019/008050

§ 371 Date: Feb. 5, 2021

(30) Foreign Application Priority Data
Aug. 13, 2018 (KR) 10-2018-0094387
Jun. 21, 2019 (KR) 10-2019-0074040

(51) Int. Cl. A61K 31/7048; A61K 36/258; A61K 31/7016; A61K 8/9789; A23L 33/105; A61P 39/06; A61Q 19/00

(52) U.S. Cl. CPC A61K 31/7048; A61K 36/258; A61K 31/7016; A61K 8/63; A23L 33/105; A61P 39/06; A61Q 19/00; A61K 8/9789

(57) ABSTRACT

The present specification relates to a composition comprising (20S,24R)-6-O-β-D-glucopyranosyl(1->2)-β-D-glucopyranoside-dammar-3-one-20,24-epoxy-6a,12b,25-triol, which is a novel ginsenoside, and pharmaceutically acceptable salts thereof, a hydrate thereof or a solvate thereof as an active ingredient. The composition exhibits an excellent antioxidant effect.

### TECHNICAL FIELD

The present disclosure describes a novel ginsenoside and a composition comprising the same.

### BACKGROUND ART

Ginseng (Panax ginseng C. A. Meyer) is a plant belonging to the genus Panax of the family Araliaceae, which has been used as herbal medicine in Korea, China, Japan, etc. for over 2,000 years. Saponins, polysaccharides, peptides, sitosterols, polyacetylenes and fatty acids are known as representative physiologically active ingredients of ginseng. The saponins of ginseng are called ginsenosides. As the efficacy and effect of ginseng, the action of the central nervous system, anticarcinogenic action and anticancer activity, immunomodulatory action, antidiabetic action, liver function-improving effect, cardiovascular disorder-improving and anti-arteriosclerotic action, blood pressure-regulating action, effect on improvement of menopausal disorder and osteoporosis, anti-stress and anti-fatigue actions, antioxidant activity, antiaging effect, etc. are known. The contents and compositions of ginsenoside vary greatly depending on the part of ginseng, such as root, leaf, berry, flower, seed, etc. However, the above-described known effects are mainly those of ginseng root, i.e., the root part of ginseng, and researches on the parts other than ginseng root are insufficient.

It is known that reactive oxygen species (free radicals) produced due to various physical, chemical and environmental factors such as the in-vivo enzyme system, reduction metabolisms, chemicals, pollutants, photochemical reactions, etc. cause cellular aging or various diseases by non-selectively and irreversibly damaging lipids, proteins, sugars, DNAs, etc. which are cellular components. Therefore, it is necessary to develop antioxidants capable of inhibiting cellular oxidation by removing the reactive oxygen species.

### REFERENCES OF RELATED ART

### Patent Documents

Korean Patent Publication No. 10-2016-0086149.

### DISCLOSURE

### Technical Problem

In an aspect, the present disclosure is directed to providing a composition comprising a novel ginsenoside having superior antioxidant effect.

### Technical Solution

In an aspect, the present disclosure provides an antioxidant composition comprising

(20S,24R)-6-O-β-D-glucopyranosyl(1->2)-β-D-glucopyranoside-dammar-3-one-20,24-epoxy-6a,12b,25-triol, a pharmaceutically acceptable salt thereof, a hydrate thereof or a solvate thereof as an active ingredient.

In another aspect, the present disclosure provides a use of (20S,24R)-6-O-β-D-glucopyranosyl(1->2)-β-D-glucopyranoside-dammar-3-one-20,24-epoxy-6a,12b,25-triol, a pharmaceutically acceptable salt thereof, a hydrate thereof or a solvate thereof for preparation of an antioxidant composition.

In another aspect, the present disclosure provides an antioxidant method comprising administering an effective amount of (20S,24R)-6-O-β-D-glucopyranosyl(1->2)-β-D-glucopyranoside-dammar-3-one-20,24-epoxy-6a,12b,25-triol, a pharmaceutically acceptable salt thereof, a hydrate thereof or a solvate thereof to a subject.

In another aspect, the present disclosure provides (20S,24R)-6-O-β-D-glucopyranosyl(1->2)-β-D-glucopyranoside-dammar-3-one-20,24-epoxy-6a,12b,25-triol, a pharmaceutically acceptable salt thereof, a hydrate thereof or a solvate thereof as an active ingredient for use in preparation of an antioxidant composition. In addition, the present disclosure provides a non-therapeutic use of (20S,24R)-6-O-β-D-glucopyranosyl(1->2)-β-D-glucopyranoside-dammar-3-one-20,24-epoxy-6a,12b,25-triol, a pharmaceutically acceptable salt thereof, a hydrate thereof or a solvate thereof as an active ingredient for antioxidation.

### Advantageous Effects

In an aspect, the present disclosure may provide a composition having superior antioxidant effect by comprising a novel ginsenoside, a pharmaceutically acceptable salt thereof, a hydrate thereof or a solvate thereof. The novel ginsenoside exhibits remarkably superior antioxidant effect as compared to the existing ginsenosides known to have antioxidant effect.

### BRIEF DESCRIPTION OF DRAWINGS

FIG. 1 shows a process of isolating a novel ginsenoside of the present disclosure (Cpd. 10) from among the compounds fractionated from a ginseng seed extract.

FIG. 2A shows the chemical structure of Compounds 1-3 fractionated from a ginseng seed extract.

FIG. 2B shows the chemical structure of Compounds 4-6 fractionated from a ginseng seed extract.

FIG. 2C shows the chemical structure of Compound 7 fractionated from a ginseng seed extract.

FIG. 2D shows the chemical structure of Compound 8 fractionated from a ginseng seed extract.

FIG. 2E shows the chemical structure of Compound 9 fractionated from a ginseng seed extract.

FIG. 2F shows the chemical structure of Compound 10 fractionated from a ginseng seed extract.

FIG. 2G shows the chemical structure of Compound 11 fractionated from a ginseng seed extract.

FIG. 2H shows the chemical structure of Compound 12 fractionated from a ginseng seed extract.

FIG. 2I shows the chemical structure of Compound 13 fractionated from a ginseng seed extract.

FIG. 2J shows the chemical structure of Compound 14 fractionated from a ginseng seed extract.

FIG. 2K shows the chemical structure of Compound 15 fractionated from a ginseng seed extract.

FIG. 2L shows the chemical structure of Compound 16 fractionated from a ginseng seed extract.

FIG. 3A shows the spectroscopic evidence and structure of Compound 1, which is a previously known ginsenoside fractionated from a ginseng seed extract.

FIG. 3B shows the spectroscopic evidence and structure of Compound 2, which is a previously known ginsenoside fractionated from a ginseng seed extract.

FIG. 3C shows the spectroscopic evidence and structure of Compound 3, which is a previously known ginsenoside fractionated from a ginseng seed extract.

FIG. 3D shows the spectroscopic evidence and structure of Compound 4, which is a previously known ginsenoside fractionated from a ginseng seed extract.

FIG. 3E shows the spectroscopic evidence and structure of Compound 5, which is a previously known ginsenoside fractionated from a ginseng seed extract.

FIG. 3F shows the spectroscopic evidence and structure of Compound 6, which is a previously known ginsenoside fractionated from a ginseng seed extract.

FIG. 4 shows the ¹H-NMR spectrum of Compound 10, which is a novel ginsenoside of the present disclosure fractionated from a ginseng seed extract.

FIG. 5 shows the ¹³C-NMR spectrum of Compound 10, which is a novel ginsenoside of the present disclosure fractionated from a ginseng seed extract.

FIG. 6 shows the COSY spectrum of Compound 10, which is a novel ginsenoside of the present disclosure fractionated from a ginseng seed extract.

FIG. 7 shows the HSQC spectrum of Compound 10, which is a novel ginsenoside of the present disclosure fractionated from a ginseng seed extract.

FIG. 8 shows the HMBC spectrum of Compound 10, which is a novel ginsenoside of the present disclosure fractionated from a ginseng seed extract.

FIG. 9 shows the MS spectrum of Compound 10, which is a novel ginsenoside of the present disclosure fractionated from a ginseng seed extract.

FIG. 10 shows the key HMBC correlation of Compound 10, which is a novel ginsenoside of the present disclosure fractionated from a ginseng seed extract.

FIG. 11 shows a result of comparing the DPPH inhibition (%) of Compounds 1-6 (GS #01-06), which are previously known ginsenosides fractionated from a ginseng seed extract, and Compound 10 (GS #10), which is a novel ginsenoside of the present disclosure (*** P<0.001 vs. (−), ** P<0.01 vs (−), * P<0.05 vs. (−)).

FIG. 12 shows a result of comparing the reactive oxygen species-scavenging ability of Compounds 1-6 (GS #01-06), which are previously known ginsenosides fractionated from a ginseng seed extract, and Compound 10 (GS #10), which is a novel ginsenoside of the present disclosure (*** P<0.001 vs. LPS, ** P<0.01 vs. LPS, * P<0.05 vs. LPS).

FIG. 13 shows a result of comparing the DPPH inhibition (%) of ginsenosides Rg1, Rg3 and Rb1, which are marker compounds of red ginseng, and Compound 10 (GS #10), which is a novel ginsenoside of the present disclosure, at different concentrations (1 μM, 10 μM) (*** P<0.001 vs. (−), ** P<0.01 vs (−), * P<0.05 vs. (−)).

FIG. 14 shows a result of comparing the reactive oxygen species-scavenging ability of ginsenosides Rg1, Rg3 and Rb1, which are marker compounds of red ginseng, and Compound 10 (GS #10), which is a novel ginsenoside of the present disclosure, at different concentrations (1 μM, 10 μM) (*** P<0.001 vs. LPS, ** P<0.01 vs. LPS, * P<0.05 vs. LPS).

FIG. 15 shows the cell survival rate (% viable cells) of Compound 10 (GS #10), which is a novel ginsenoside of the present disclosure (*** P<0.001 vs. (−), ** P<0.01 vs (−), * P<0.05 vs. (−)).

### BEST MODE

Hereinafter, exemplary embodiments of the present disclosure will be described in more detail referring to the attached drawings. However, the present disclosure may be embodied in different forms without being limited to the exemplary embodiments described herein. Rather, the exemplary embodiments are provided so that this disclosure will be thorough and complete, and will fully convey the technical idea of the present disclosure to those skilled in the art. In the drawings, the sizes of some elements such as width, thickness, etc. are somewhat exaggerated in order to clarify the elements. In addition, although some elements are shown only in part for the convenience of explanation, those skilled in the art will easily understand the remaining part of the elements. In addition, those having ordinary knowledge in the art will be able to embody the technical idea of the present disclosure in various other forms without departing from the scope of the present disclosure.

In an exemplary embodiment, the present disclosure may provide an antioxidant composition comprising a novel ginsenoside, a pharmaceutically acceptable salt thereof, a hydrate thereof or a solvate thereof as an active ingredient.

In an exemplary embodiment, the ginsenoside is (20S,24R)-6-O-β-D-glucopyranosyl(1->2)-β-D-glucopyranoside-dammar-3-one-20,24-epoxy-6a,12b,25-triol, which is a novel triterpene saponin.

In an exemplary embodiment, the present disclosure may provide a use of (20S,24R)-6-O-β-D-glucopyranosyl(1->2)-β-D-glucopyranoside-dammar-3-one-20,24-epoxy-6a,12b,25-triol, a pharmaceutically acceptable salt thereof, a hydrate thereof or a solvate thereof for preparation of an antioxidant composition.

In an exemplary embodiment, the present disclosure may provide an antioxidant method comprising administering an effective amount of (20S,24R)-6-O-β-D-glucopyranosyl(1->2)-β-D-glucopyranoside-dammar-3-one-20,24-epoxy-6a,12b,25-triol, a pharmaceutically acceptable salt thereof, a hydrate thereof or a solvate thereof to a subject.

In an exemplary embodiment, the present disclosure may provide (20S,24R)-6-O-β-D-glucopyranosyl(1->2)-β-D-glucopyranoside-dammar-3-one-20,24-epoxy-6a,12b,25-triol, a pharmaceutically acceptable salt thereof, a hydrate thereof or a solvate thereof as an active ingredient for use in an antioxidant composition. In addition, the present disclosure may provide a non-therapeutic cosmetic use of (20S,24R)-6-O-β-D-glucopyranosyl(1->2)-β-D-glucopyranoside-dammar-3-one-20,24-epoxy-6a,12b,25-triol, a pharmaceutically acceptable salt thereof, a hydrate thereof or a solvate thereof as an active ingredient for antioxidation.

As used herein, the term “pharmaceutically acceptable” refers to those that can be approved or was approved by the government or equivalent regulatory agencies for use in animals, more specifically in humans, by avoiding significant toxic effects when used in conventional medicinal dosage, or those recognized as being listed in the pharmacopoeia or described in other general pharmacopoeia.

As used herein, the term “pharmaceutically acceptable salt” refers to a salt according to one aspect of the present disclosure that is pharmaceutically acceptable and possesses the desired pharmacological activity of the parent compound. The salts comprise (1) acid addition salts, formed with inorganic acids such as hydrochloric acid, hydrobromic acid, sulfuric acid, nitric acid, phosphoric acid, or the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl) benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2,2,2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, or the like; or (2) salts formed when an acidic proton present in the parent compound is substituted.

As used herein, a “hydrate” refers to a compound bound with water. It is used in a broad sense, comprising an inclusion compound which lacks chemical bonding with water.

As used herein, a “solvate” refers to a higher-order compound formed between a solute molecule or ion and a solvent molecule or ion.

In an exemplary embodiment, the ginsenoside has a molecular formula of C₄₂H₇₀O₁₅ and has the following chemical structure.

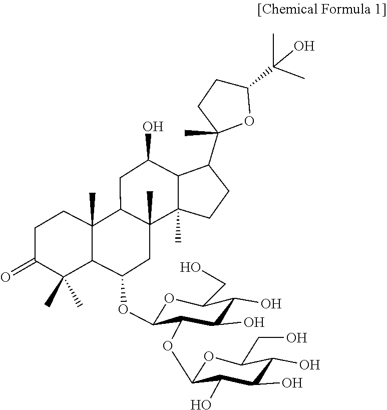

In the present disclosure, the novel ginsenoside is named “pseudoginsenoside RTs” or “PG-RT₈”.

In an exemplary embodiment, the ginsenoside may be one extracted from ginseng seed. More specifically, the ginsenoside may be one isolated from a ginseng seed extract, although not being limited thereto. In an exemplary embodiment, the ginseng seed may be the seed of Panax ginseng C. A. Meyer.

In the present disclosure, “isolation” means extraction or fractionation from a ginseng seed extract using water or an organic solvent by any method known to those skilled in the art. The fractionation may be performed after the extraction.

As used herein, the term “extract” means a substance obtained by extracting a component contained inside of a natural substance, regardless of the extracted method or ingredients. The term is used in a broad sense comprising, for example, all of those obtained by extracting a component soluble in a solvent from a natural substance using water or an organic solvent, extracting only a specific component of a natural substance, or the like.

In the present disclosure, the “fraction” comprises a fraction obtained by fractionating a specific substance or extract using a solvent, a remainder remaining after the fractionation, or a fraction obtained by extracting again using a specific solvent. The fractionation or extraction may be conducted by any methods known to those of ordinary skill in the art.

In an exemplary embodiment, the ginsenoside may be one isolated from a methanol- and butanol-soluble extract of ginseng seed. Specifically, the ginsenoside may be detected and isolated from a methanol- and butanol-soluble extract of ginseng seed by HPLC-ESI-Q-TOF-MS. Since the main components of the ginseng seed extract are lipids, not all triterpene and steroidal saponins can be observed from a crude extract of ginseng seed by HPLC-UV or HPLC-ELSD.

In an exemplary embodiment, the present disclosure may provide a composition inhibiting the activity of 2,2-diphenyl-1-picrylhydrazyl (DPPH), which comprises the said ginsenoside, a pharmaceutically acceptable salt thereof, a hydrate thereof or a solvate thereof. In an exemplary embodiment, the present disclosure may provide a composition removing reactive oxygen species (ROS), which comprises the ginsenoside, a pharmaceutically acceptable salt thereof, a hydrate thereof or a solvate thereof.

In an exemplary embodiment, the present disclosure may provide a composition having remarkably superior antioxidant effect as compared to existing ginsenosides known to have antioxidant effect.

In an exemplary embodiment, the active ingredient may be comprised in an amount of 0.0001-99.9 wt % based on the total weight of the composition. Specifically, in an exemplary embodiment, the composition may comprise the active ingredient in an amount of 0.0001 wt % or more, 0.0005 wt % or more, 0.001 wt % or more, 0.01 wt % or more, 0.1 wt % or more, 1 wt % or more, 2 wt % or more, 3 wt % or more, 4 wt % or more, 5 wt % or more, 6 wt % or more, 7 wt % or more, 8 wt % or more, 9 wt % or more, 10 wt % or more, 15 wt % or more, 20 wt % or more, 25 wt % or more, 30 wt % or more, 35 wt % or more, 40 wt % or more, 45 wt % or more, 50 wt % or more, 55 wt % or more, 60 wt % or more, 65 wt % or more, 70 wt % or more, 75 wt % or more, 80 wt % or more, 85 wt % or more, 90 wt % or more, 95 wt % or more or 99.9 wt % or more based on the total weight of the composition, although not being limited thereto. In addition, in an exemplary embodiment, the composition may comprise the active ingredient in an amount of 100 wt % or less, 99 wt % or less, 95 wt % or less, 90 wt % or less, 85 wt % or less, 80 wt % or less, 75 wt % or less, 70 wt % or less, 65 wt % or less, 60 wt % or less, 55 wt % or less, 50 wt % or less, 45 wt % or less, 40 wt % or less, 35 wt % or less, 30 wt % or less, 25 wt % or less, 20 wt % or less, 15 wt % or less, 10 wt % or less, 9 wt % or less, 8 wt % or less, 7 wt % or less, 6 wt % or less, 5 wt % or less, 4 wt % or less, 3 wt % or less, 2 wt % or less, 1 wt % or less, 0.5 wt % or less, 0.1 wt % or less, 0.01 wt % or less, 0.001 wt % or less or 0.0005 wt % or less based on the total weight of the composition, although not being limited thereto.

The composition according to an exemplary embodiment of the present disclosure may be composition for external application to skin, which comprises the active ingredient.

In the present disclosure, “skin” refers to the tissue covering the outer surface of an animal. The term is used in the broadest concept, comprising not only the tissue covering the outer surface such as face, body, etc., but also the scalp and hair.

The composition according to exemplary embodiments of the present disclosure may be a cosmetic composition comprising the active ingredient.

In an exemplary embodiment, the composition may be formulated by comprising a cosmetically or dermatologically acceptable medium or base. It may be prepared into any formulation suitable for topical application, e.g., a solution, a gel, a solid, an anhydrous paste, an oil-in-water emulsion, a suspension, a microemulsion, a microcapsule, a microgranule, an ionic (liposome) or nonionic vesicular dispersant, a cream, a toner, a lotion, a powder, an ointment, a spray or a conceal stick. Also, it may be used in the form of a foam or an aerosol composition further comprising a compressed propellant. The composition may be prepared according to a method commonly employed in the art.

The composition according to exemplary embodiments of the present disclosure may be a food composition comprising the active ingredient.

For example, the composition may be processed into a functional food comprising the active ingredient, such as fermented milk, cheese, yogurt, juice, a probiotic, a health food, etc. and may also be used in the form of various food additives. In an exemplary embodiment, the composition may be a health food composition. In an exemplary embodiment, the health food composition may be formulated as a pill, a capsule, a tablet, a granule, a caramel, a drink, etc. In another exemplary embodiment, it may be processed into such forms as a liquid, a powder, a granule, a tablet, a tea bag, etc. The composition may be administered by various methods such as simple drinking, administration by injection, spraying, squeezing, etc. The composition may comprise other ingredients, etc. that may provide a synergistic effect to a main effect within a range not negatively affecting the main effect of the present disclosure. For example, it may further comprise an additive such as a flavorant, a colorant, a sterilizer, an antioxidant, an antiseptic, a moisturizer, a thickener, a mineral, an emulsifier, a synthetic polymer, etc. for improvement of physical properties. In addition, it may further comprise an auxiliary ingredient such as a water-soluble vitamin, an oil-soluble vitamin, a polypeptide, a polysaccharide, a seaweed extract, etc. These ingredients may be selected and mixed adequately by those skilled in the art depending on the formulation or purpose of use, and the addition amount thereof may be selected within ranges not negatively affecting the purpose and effect of the present disclosure. For example, the addition amount of these ingredients may be 0.0001-99.9 wt % based on the total weight of the composition.

The composition according to exemplary embodiments of the present disclosure may be a pharmaceutical composition comprising the active ingredient. The pharmaceutical composition may further comprise a pharmaceutical adjuvant such as an antiseptic, a stabilizer, a wetting agent, an emulsification accelerator, a salt and/or buffer for adjusting osmotic pressure, etc. and other therapeutically useful materials.

In an exemplary embodiment, the pharmaceutical composition may be a composition for oral administration. For example, the composition for oral administration may be a tablet, a pill, a hard or soft capsule, a liquid, a suspension, an emulsion, a syrup, a powder, a dust, a fine granule, a granule, a pellet, etc. These formulations may further comprise, in addition to the active ingredient, a surfactant, a diluent (e.g., lactose, dextrose, sucrose, mannitol, sorbitol, cellulose and glycine) and a lubricant (e.g., silica, talc, stearic acid and its magnesium or calcium salts, and polyethylene glycol). A tablet may further comprise a binder such as magnesium aluminum silicate, starch paste, gelatin, tragacanth, methyl cellulose, sodium carboxymethyl cellulose or polyvinylpyrrolidone. If necessary, the tablet may further comprise other pharmaceutical additives, for example, a disintegrant such as starch, agar, alginic acid or a sodium salt thereof, an adsorbent, a coloring agent, a flavorant, a sweetener, etc. The tablet may be prepared by a common mixing, granulation or coating method.

In an exemplary embodiment, the pharmaceutical composition may be a composition for parenteral administration, and the composition for parenteral administration may be a formulation for rectal, topical, subcutaneous or transdermal administration. For example, the formulation may be an injection, a medicinal drop, an ointment, a lotion, a gel, a cream, a spray, a suspension, an emulsion, a suppository, a patch, etc., although not being limited thereto.

In an exemplary embodiment, the administration amount of the pharmaceutical composition will vary depending on the age, sex and body weight of the subject to be treated, the particular disease or pathological condition to be treated, the severity of the disease or pathological condition, and the discretion of a prescriber. The determination of the administration amount based on these factors is within the level of those skilled in the art. For example, the administration dosage may be in a range from 1 mg/kg/day to 10 g/kg/day, or from 5 mg/kg/day to 100 mg/kg/day. However, the administration dosage does not limit the scope of the present disclosure by any means.

### MODE FOR INVENTION

Hereinafter, the present disclosure will be described in detail referring to examples, comparative examples and test examples. However, the following examples are for illustrative purposes only and it will be obvious to those or ordinary skill in the art that the scope of the present disclosure is not limited by the examples, comparative examples and test examples.

All the experimental values given below are averages of at least three repeated experiments and standard deviation (SD) is represented by error bars. p values were calculated by one-way ANOVA and Dunnett's test, and p values smaller than 0.05 were considered statistically significant.

### [Example 1] Isolation of Ginsenosides

Fractionation

5.5 kg of Ginseng seed (seeds of Panax ginseng) was finely ground with a mixer to make a powder form, which was extracted with methanol and then fractionated step by step using n-hexane, ethyl acetate, n-butanol, etc. Lipids were mostly removed by n-hexane, and the lipids remaining in the ethyl acetate fraction were suspended in methanol:water (=1:1 (v/v)), stored in a freezer overnight, and then only the supernatant was taken. The lipids were removed once more using a centrifuge. 2.61 g of the ethyl acetate fraction and 114.64 g of the n-butanol fraction thus pretreated were fractionated through column and high-performance counter-current chromatography (HPCCC) as follows.

Fractionation of n-Butanol Fraction Using Column and HPCCC

114.64 g of the n-butanol fraction was fractionated by MPLC. n-Hexane/ethyl acetate (=10:1→5:1→1:1) and CHCl₃/MeOH (=10:1→5:1 (v/v)) were used as solvents and the flow rate was 50 mL/min. A total of 12 subfractions were obtained under the above conditions, and the components contained in each fraction were separated again using HPCCC, high-performance liquid chromatography (HPLC), Sephadex LH-20 column, etc. Subsequently, 16 compounds were identified by investigating their structure using nuclear magnetic resonance (NMR), ultraviolet (UV) spectroscopy and mass spectrometry (MS).

The isolated 16 compounds comprise: ginsenoside Rg1 (Compound 1), ginsenoside Rg2 (Compound 2) and ginsenoside Re (Compound 3), which are protopanaxatriol saponins; ginsenoside Rd (Compound 4), ginsenoside Rb1 (Compound 5) and ginsenoside Rb2 (Compound 6), which are protopanaxadiol saponins; stigma-5-en-3-O-β-D-glucopyranoside (Compound 7), stigma-5,24(28)-dien-3-O-β-D-glucopyranoside (Compound 8) and stigma-5,22-dien-3-O-β-D-glucopyranoside (Compound 9), which are sterol glycosides;

(20S,24R)-6-O-β-D-glucopyranosyl(1->2)-β-D-glucopyranoside-dammar-3-one-20,24-epoxy-6a,12b,25-triol (Compound 10), which is a novel ginsenoside compound according to an exemplary embodiment of the present disclosure first isolated from a natural product; phenethyl alcohol β-D-xylopyranosyl(1->6)-β-D-glucopyranoside) (Compound 12) and eugenyl β-gentiobioside (Compound 13), which are phenolic glycosides; isorhamnetin 3-O-β-D-glucopyranoside (Compound 15), which is a flavonoid; and adenosine (Compound 11), uracil (Compound 14) and tryptophan (Compound 16), which are primary metabolites.

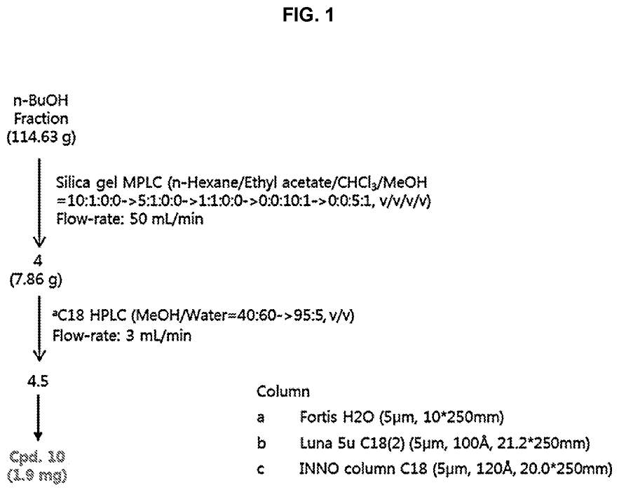

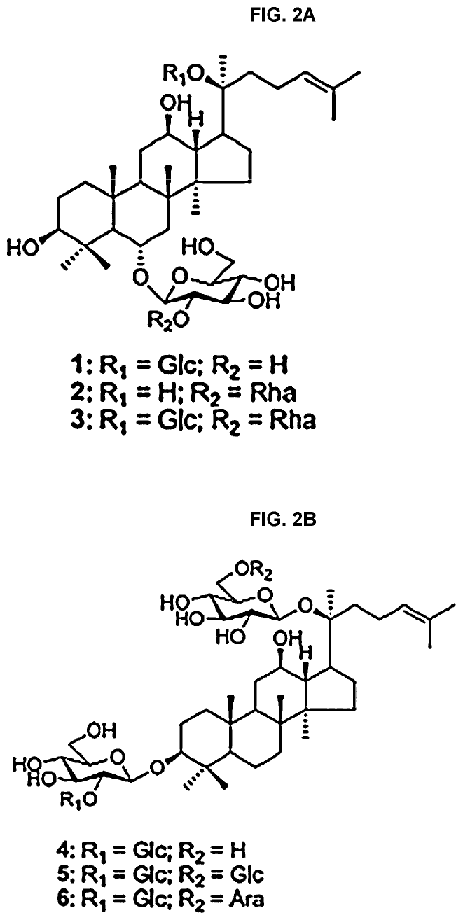

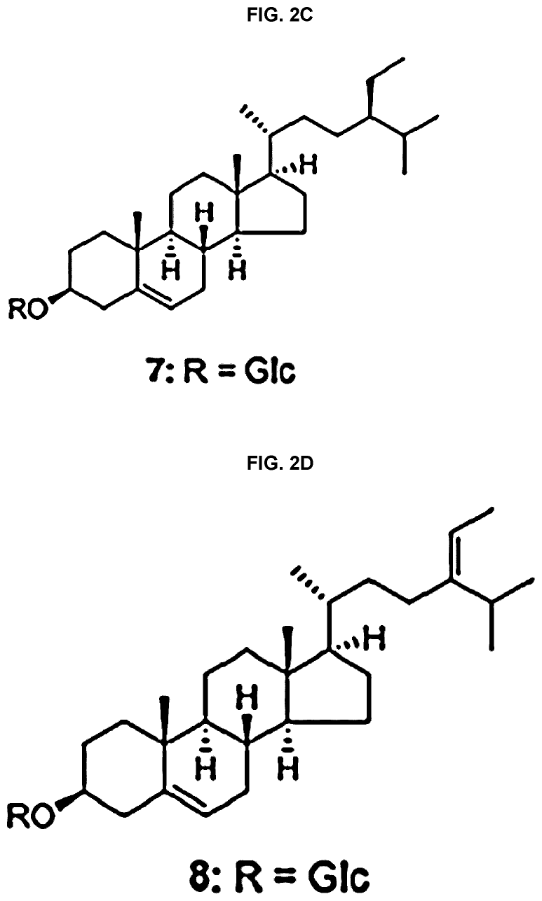

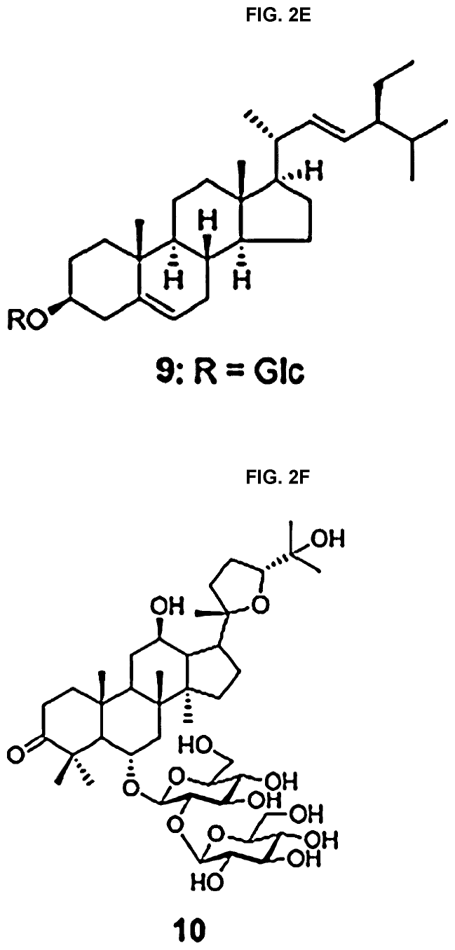

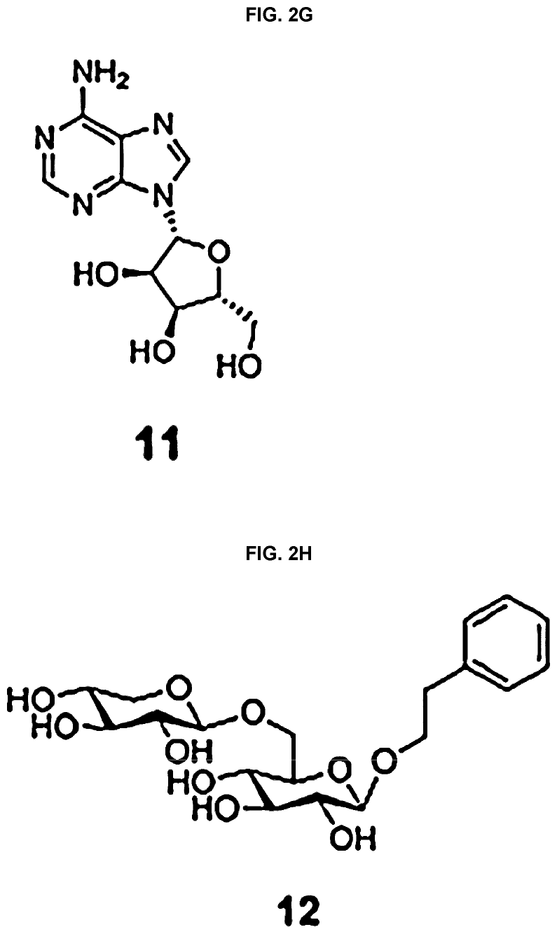

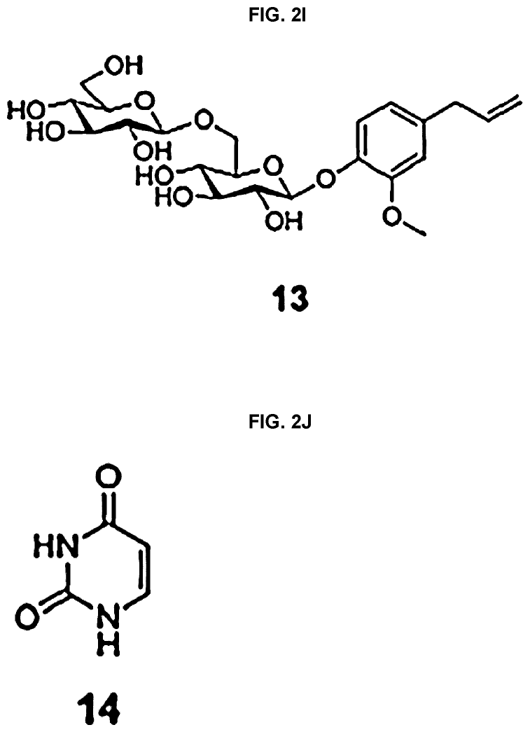

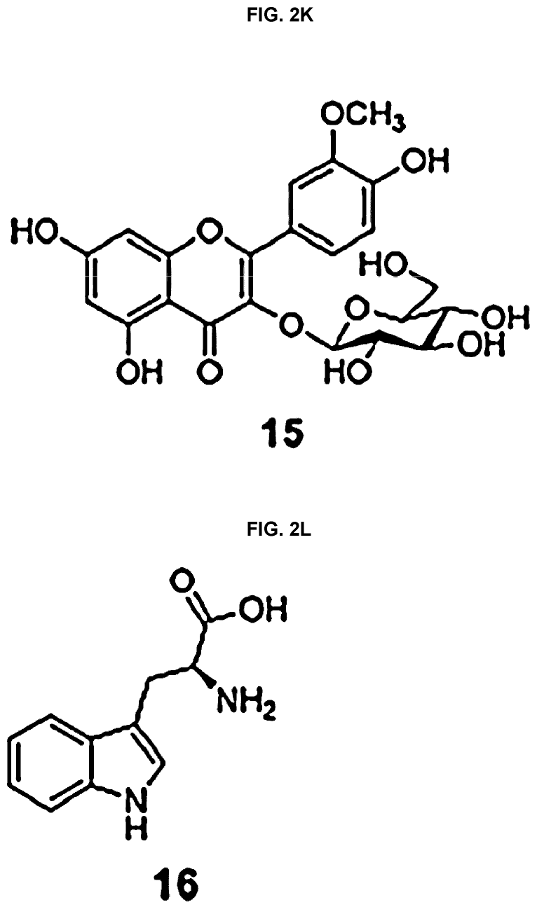

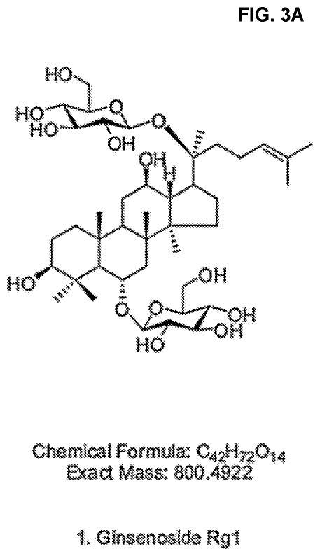

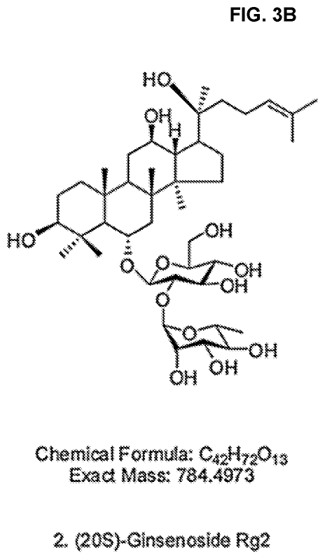

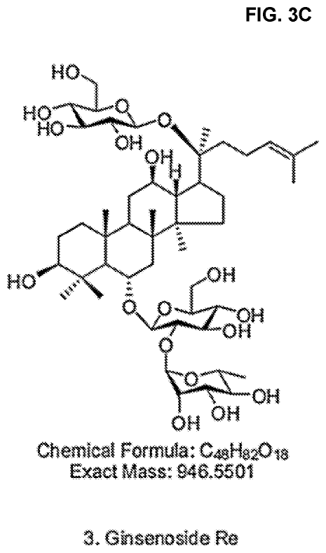

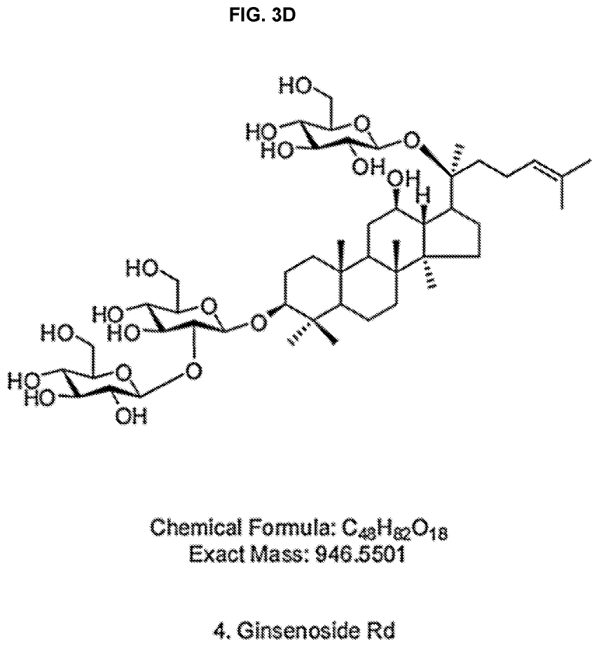

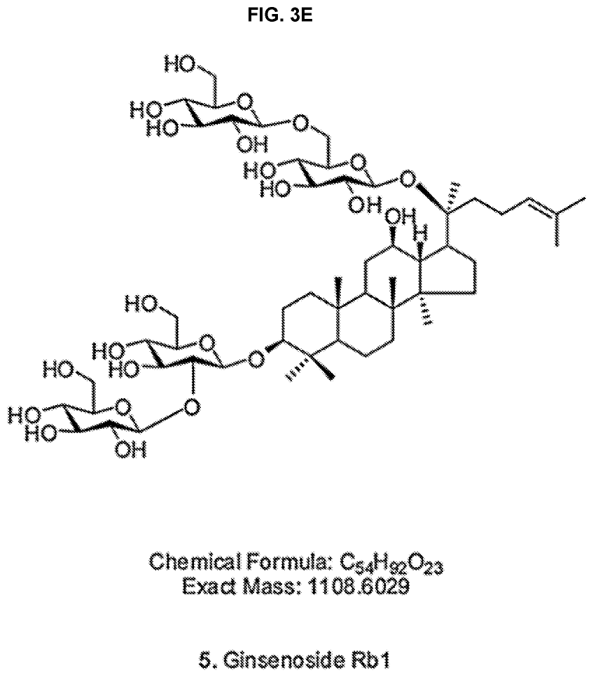

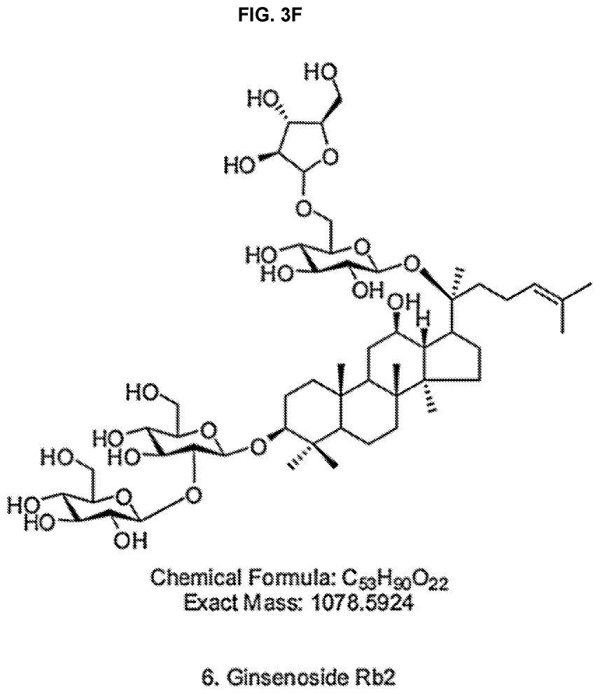

The isolation process of Compound 10, which is a novel ginsenoside according to an embodiment of the present disclosure, is shown in FIG. 1. The chemical structures of the 16 compounds are shown in FIGS. 2A-2L, and the spectroscopic evidence and chemical structures of the previously known ginsenosides (Compounds 1-6) among the above compounds are additionally shown in FIGS. 3A-3F.

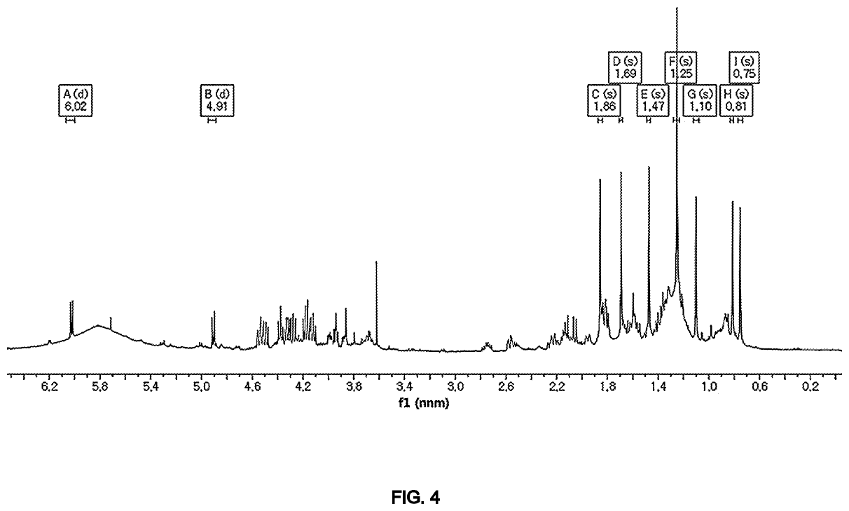

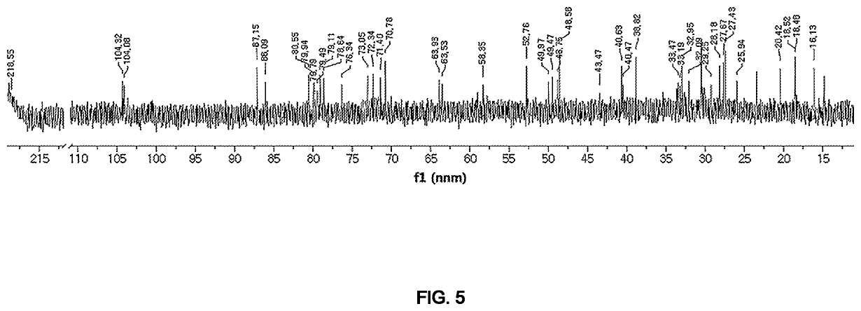

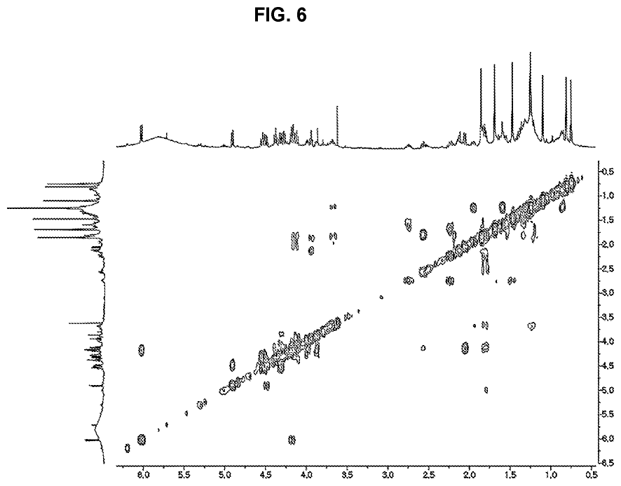

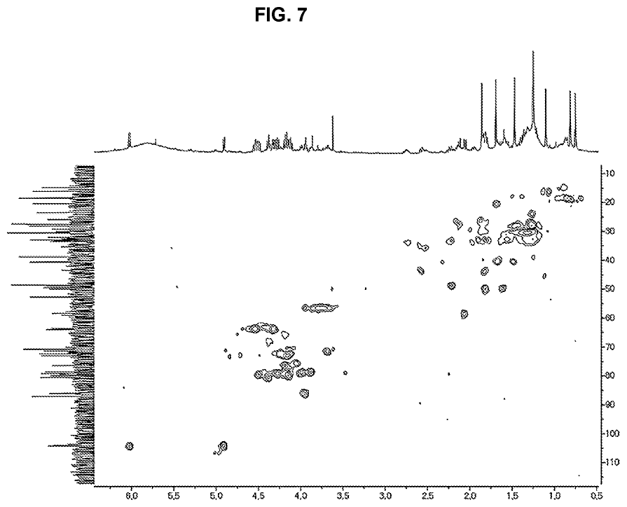

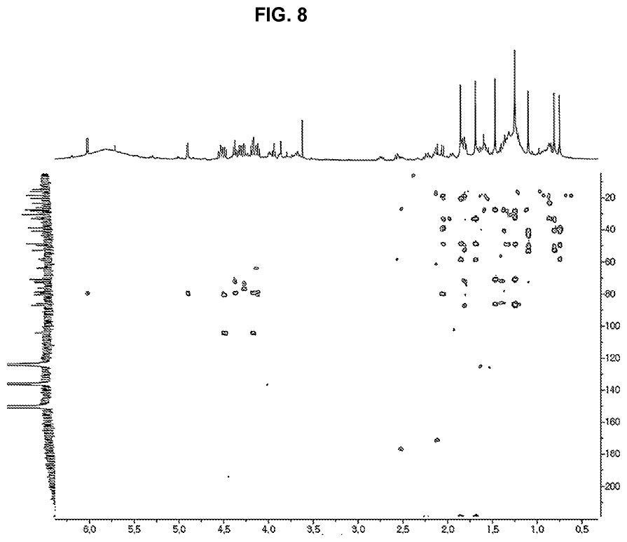

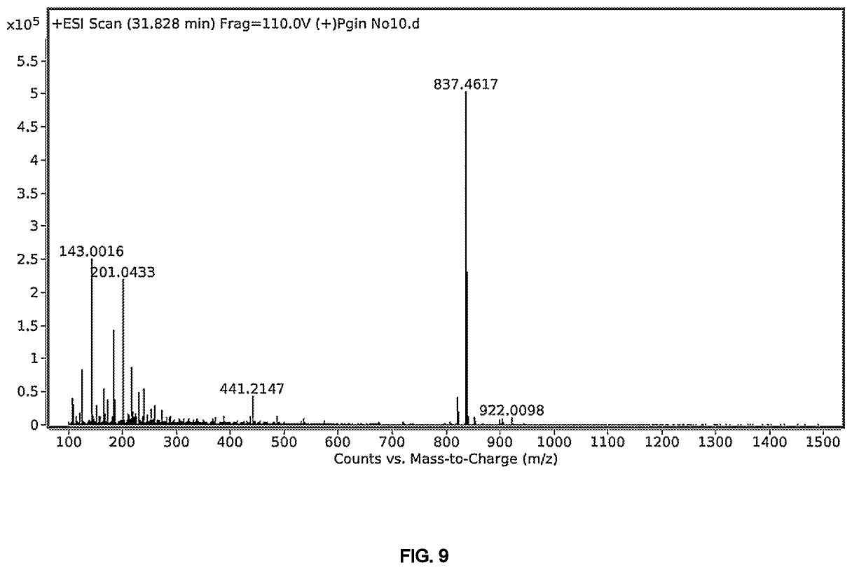

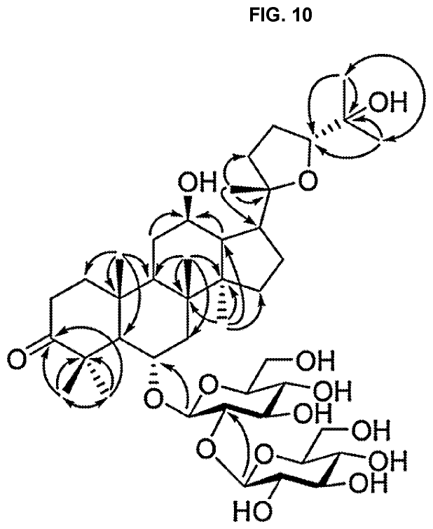

Compound 10 was isolated as a white amorphous powder with the molecular formula C₄₂H₇₀O₁₅ based on the sodiated pseudomolecular ion peak at m/z 837.4617 [(M+Na)⁺ calcd. 837.4612] in the cationic electrospray ionization-quadrupole-time-of-flight mass spectrometry (ESI-Q-TOF-MS) spectrum. The ¹H NMR spectrum of Compound 10 comprises 8 methyl resonance peaks [δ_{H}1.86 (3H, s, H-28), 1.69 (3H, s, H-29), 1.47 (3H, s, H-27), 1.25 (6H, s, H-21, 26), 1.10 (3H, s, H-18), 0.81 (3H, s, H-30), 0.75 (3H, s, H-19)]. In addition, two pairs of signals corresponding to anomeric protons and carbon atoms at two sugar residues were detected at δ_{H} 6.02 (1H, d, J=7.8, H-2″)/δ_{C} 104.08 (C-1′) and δ_{H} 4.91 (1H, d, J=7.7, H-1′)/δ_{C} 104.32 (C-1″). The ¹³C NMR and heteronuclear single quantum correlation (HSQC) spectra revealed 42 carbon signals. Apart from the above two sugar residues, the aglycone of Compound 10 had eight methylenes, four methines, three oxygen-containing methines [δ_{C} 79.79 (C-6), 71.40 (C-12) and 86.09 (C-24)], five quaternary carbon atoms, two oxygenated quaternary carbon atoms [δ_{C} 87.15 (C-20) and 70.78 (C-25)], eight methyl groups and carbonyl carbon [δ_{C} 218.85 (C-3)]. As a result of thorough interpretation of the ¹H and ¹³C NMR data, the aglycone of Compound 10 was found to be superimposed on pseudoginsengenin R1 [(20S,24R)-dammar-3-one-20,24-epoxy-6α,12β,25-triol])]. The absolute configuration of C-20 in Compound 10 was deduced from S to chemical shift of C-21 (δ_{C} 27.67), and the 24R configuration was determined by chemical shift of C-24 (δ_{C} 86.09) as previously published. Both sugar units were turned out to be pi-D-glucopyranosyl residues from the coupling constants of the anomeric protons in the ¹H NMR spectra and 12 carbon resonances, together with acid hydrolysis data and gas chromatography (GC) analysis results. A glycoside linkage was determined by heteronuclear multiple bond correlation (HMBC) which showed cross peaks at δ_{H} 6.02 (H-1″)/δ_{C} 79.49 (C-2′) and δ_{H} 4.91 (H-1′)/δ_{C} 79.79 (C-6), and it was demonstrated that 2-O-(β-D-glucopyranosyl-β-D-glucopyranosyl residues were linked to C-6 of aglycone at pseudoginsengenin R1. Each of the analytical spectra of Compound 10 and the core HBMC correlation are shown in FIGS. 4 to 10.

As a result of the analysis, the chemical structure of Compound 10 was determined as

(20S,24R)-6-O-β-D-glucopyranosyl(1->2)-β-D-glucopyranoside-dammar-3-one-20,24-epoxy-6a,12b,25-triol, and the compound was named pseudoginsenoside RT8 (PG-RT₈).

Among the ginsenosides isolated from the ginseng seed extract, ginsenoside Rg1 (Compound 1), ginsenoside Rg2 (Compound 2) and ginsenoside Re (Compound 3), which are protopanaxtriol (PPT)-based ginsenosides, comprise three hydroxyl groups in the ginsenoside backbone. Ginsenoside Rd (Compound 4), ginsenoside Rb1 (Compound 5) and ginsenoside Rb2 (Compound 6), which are protopanaxdiol (PPD)-based ginsenosides, comprise two hydroxyl groups in the ginsenoside backbone. On the other hand, Compound 10, which is a ginsenoside newly isolated and identified in the present disclosure, has a PPT-based backbone, but the terminal hydroxyl group of the backbone is a ketone, and there is a structural difference in that the linear chain of the ginsenoside is cyclized into a furan ring.

Compound 10, which was newly isolated and identified in the present disclosure, had a molecular formula of C₄₂H₇₀O₁₅. ESI-Q-TOF-MS m/z was 837.4617 [M+Na]⁺ and the ¹H- and ¹³C-NMR spectra data are given in the following tables.

TABLE 1

| Position | 13C-NMR | 1H-NMR |
|---|---|---|
| 1 | 40.63 | 1.67 (1H, H-1a)^{a}, 1.49 (1H, H-1b)^{a} |
| 2 | 33.61 | 2.23 (1H, H-2a)^{a}, 1.78 (1H, H-2b)^{a} |
| 3 | 218.85 | — |
| 4 | 48.58 | — |
| 5 | 58.35 | 2.06 (1H, d, J = 10.6 Hz, H-5) |
| 6 | 79.79 | 4.15 (1H, H-6)^{a} |
| 7 | 43.47 | 2.57(1H, H-7a)^{a}, 1.82 (1H, H-7b)^{a} |
| 8 | 40.47 | — |
| 9 | 49.47 | 1.60 (1H, H-9)^{a} |
| 10 | 38.82 | — |
| 11 | 33.47 | 2.22 (1H, H-11a)^{a}, 1.32 (1H, H-11b)^{a} |
| 12 | 71.40 | 3.68 (1H, td, J = 10.6, 4.5 Hz, H-12) |
| 13 | 49.97 | 1.81 (1H, H-13)^{a} |
| 14 | 52.76 | — |
| 15 | 33.19 | 1.64 (1H, H-15a)^{a}, 1.26 (1H, H-15b)^{a} |
| 16 | 25.94 | 2.17 (1H, H-16a)^{a}, 1.87 (1H, H-16b)^{a} |
| 17 | 48.75 | 2.21 (1H, H-17)^{a} |
| 18 | 16.13 | 1.10 (3H, s, H-18) |
| 19 | 18.48 | 0.75 (3H, s, H-19) |
| 20 | 87.15 | — |
| 21 | 27.67 | 1.25 (3H, s, H-21) |
| 22 | 32.09 | 1.60 (1H, H-22a)^{a}, 1.37 (1H, H-22b)^{a} |
| 23 | 29.25 | 1.82 (1H, H-23a)^{a}, 1.25 (1H, H-23b)^{a} |
| 24 | 86.09 | 3.94 (1H, t, J = 7.5 Hz, H-24) |
| 25 | 70.78 | — |
| 26 | 27.43 | 1.25 (3H, s, H-26) |
| 27 | 28.18 | 1.45 (3H, s, H-27) |
| 28 | 32.95 | 1.86 (3H, s, H-28) |
| 29 | 20.42 | 1.69 (3H, s, H-29) |
| 30 | 18.52 | 0.81 (3H, s, H-30) |

^{a}peak was overlapped

TABLE 2

| | | |
|---|---|---|
| Position | 13C-NMR | 1H-NMR |

6-O-Glc

| | | |
|---|---|---|
| 1′ | 104.08 | 4.91 (1H, d, J = 7.7 Hz, H-1′) |
| 2′ | 79.49 | 4.48 (1H, m, H-2′) |
| 3′ | 80.55 | 4.38 (1H, m, H-3′) |
| 4′ | 73.05 | 4.16 (1H, m, H-4′) |
| 5′ | 79.94 | 4.15 (1H, m, H-5′) |
| 6′ | 63.53 | 4.54 (1H, m, H-6′a), |
| | | 4.32 (1H, m, H-6′b) |

2-O-Glc

| | | |
|---|---|---|
| 1″ | 104.32 | 6.02 (1H, d, J = 7.8 Hz, H-1″) |
| 2″ | 76.34 | 4.18 (1H, m, H-2″) |
| 3″ | 78.64 | 3.99 (1H, m, H-3″) |
| 4″ | 72.34 | 4.12 (1H, m, H-4″) |
| 5″ | 79.11 | 4.27 (1H, m, H-5″) |
| 6″ | 63.93 | 4.54 (1H, m, H-6″a), |
| | | 4.32 (1H, m, H-6″b) |

### [Test Example 1] Comparison of Antioxidant Effect 1

In order to compare the antioxidant effect of the ginsenosides isolated from the ginseng seed extract, the effect of inhibiting 2,2-diphenyl-1-picrylhydrazyl (DPPH) was tested as follows.

The DPPH (2,2-diphenyl-1-picrylhydrazyl) radical dissolved in an organic solvent has maximum absorbance at 515 nm. An antioxidant substance scavenges the DPPH radical, thereby turning the solution colorless and transparent. Accordingly, antioxidant effect can be compared by measuring the DPPH radical-scavenging activity of the ginsenosides through DPPH assay.

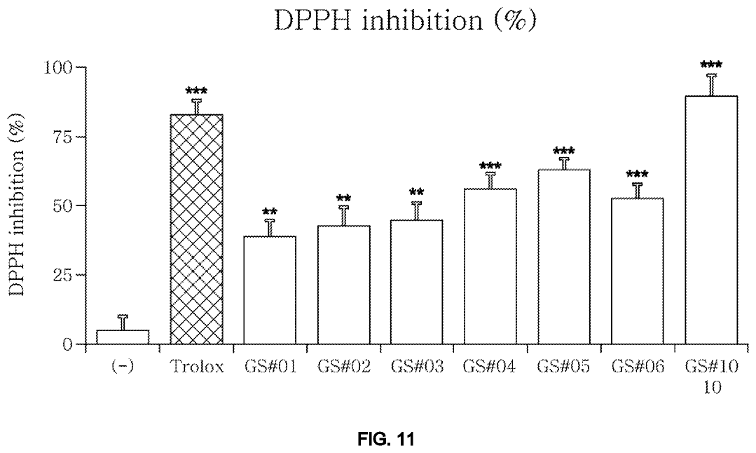

First, a DPPH solution was prepared by dissolving 4 mg of DPPH (Sigma) in 100 mL of ethanol (Sigma). Then, 10 μM of the novel ginsenoside GS #10 of an exemplary embodiment of the present disclosure or each of the ginsenosides GS #01 to GS #06 as comparative examples of the present disclosure, isolated from the ginseng seed extract, were dissolved in 200 μL of the DPPH solution. Trolox (Sigma; 10 μM) was used as a positive control group. After keeping the reaction solution at room temperature for 30 minutes, absorbance was measured at a wavelength of 515 nm. DPPH (reactive oxygen species)-scavenging ability was calculated based on the data and are shown in FIG. 11.

As seen from FIG. 11, Compound 10 (GS #10), which is the novel ginsenoside of the present disclosure, exhibited superior antioxidant effect, with remarkably higher DPPH (reactive oxygen species)-scavenging ability, i.e., DPPH-inhibiting ability, as compared to Compounds 1-6 (GS #01 to GS #06), which are previously known ginsenosides as comparative examples of the present disclosure, at the same concentration. This is due to the difference in chemical structure and means that the novel ginsenoside of the present disclosure, PG-RT₈, has excellent antioxidant effect among the ginseng seed-derived ginsenosides and exhibits stronger antioxidant activity than the previously known steroidal saponins.

### [Test Example 2] Comparison of Antioxidant Effect 2

In order to compare the antioxidant effect of the ginsenosides isolated from the ginseng seed extract, the effect of scavenging reactive oxygen species (ROS) was tested as follows.

RAW 264.7 macrophages purchased from ATCC were cultured in Dulbecco's modified Eagle's medium (Sigma) supplemented with 10% fetal bovine serum (FBS; HyClone) and 1% penicillin/streptomycin (Sigma) in a 5% CO₂ incubator. After treating the RAW 264.7 cells with NAC (N-acetylcysteine; positive control group; Sigma) or each of the 7 ginsenosides extracted from the ginseng seed (GS #01 to GS #06 and GS #10; 10 μM each) for 1 hour, lipidpolysaccharides (LPS; inflammation/oxidative stress-induced group, Sigma) ware administered at a concentration of 10 ng/mL concentration for 30 minutes. Then, the cells were treated with 10 μM H₂DCFDA (2′,7′-dichlorodihydrofluorescein diacetate; Invitrogen) for 30 minutes for measurement of reactive oxygen species. The amount of accumulated reactive oxygen species was measured using a TECAN multiplate reader (Tecan AG, excitation 485 nm, emission 530 nm).

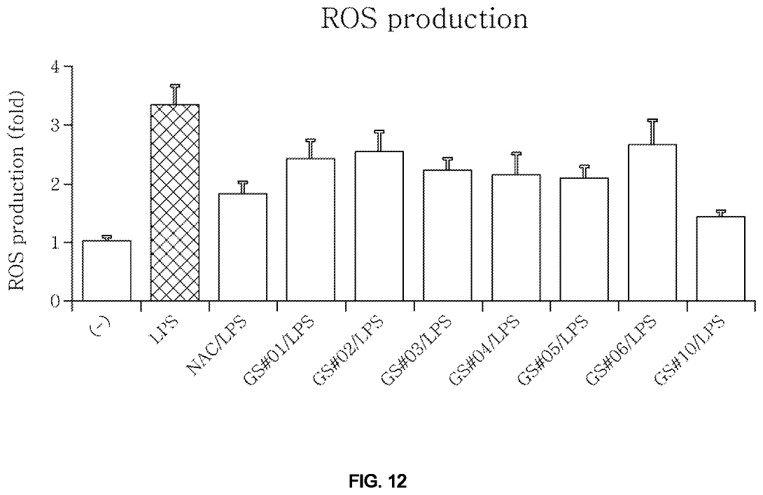

From FIG. 12, it can be seen that Compound 10 (GS #10), which is the novel ginsenoside of the present disclosure, can effectively remove the reactive oxygen species induced by LPS as compared to Compounds 1-6 (GS #01 to GS #06), which are previously known ginsenosides as comparative examples of the present disclosure, at the same concentration, confirming that it has superior antioxidant effect.

### [Test Example 3] Comparison of Antioxidant Effect 3

DPPH-inhibiting effect was compared in the same manner as in Test Example 1. The novel ginsenoside GS #10 (isolated form the ginseng seed extract) according to an exemplary embodiment of the present disclosure was compared with three marker compounds of red ginseng (Rg1, Rg3 and Rb1; Sigma) as comparative examples of the present disclosure. The concentrations of the ginsenosides were 1 and 10 μM. The chemical structure of ginsenoside Rg3, which is a comparative example of the present disclosure, is as follows.

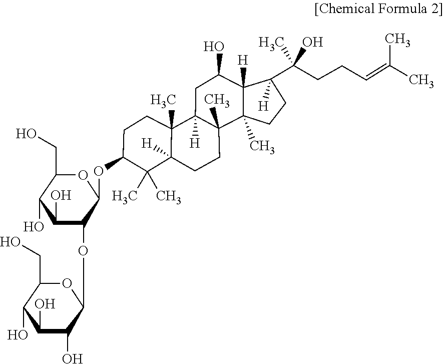

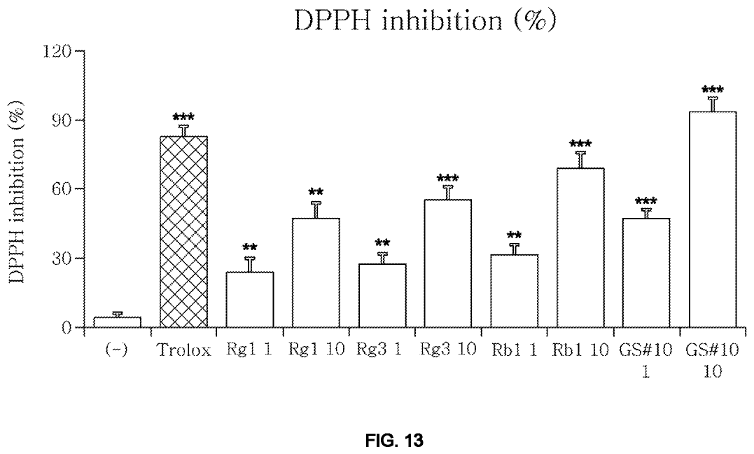

As seen from FIG. 13, the novel ginsenoside GS #10 according to an exemplary embodiment of the present disclosure showed remarkably excellent DPPH-inhibiting effect as compared to the three ginsenosides, which are marker compounds of red ginseng.

### [Test Example 4] Comparison of Antioxidant Effect 4

The reactive oxygen species-scavenging ability of the novel ginsenoside GS #10 (isolated from the ginseng seed extract) according to an exemplary embodiment of the present disclosure was compared with that of three ginsenosides (Rg1, Rg3 and Rb1; Sigma), which are marker compounds of red ginseng as comparative examples of the present disclosure, in the same manner as in Test Example 2. The concentrations of the ginsenosides were 1 and 10 μM.

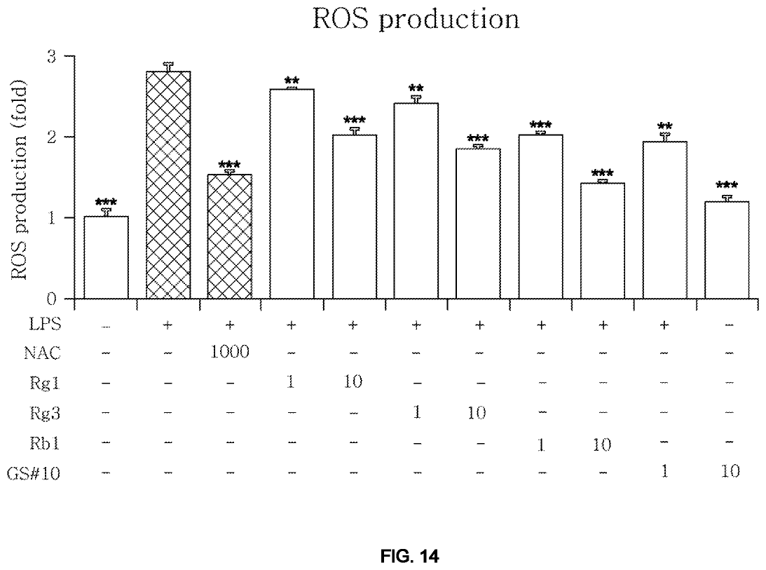

As seen from FIG. 14, the production of reactive oxygen species was decreased as the concentration of the novel ginsenoside GS #10 according to the present disclosure was increased. In particular, the reactive oxygen species-scavenging ability was remarkably superior as compared to the ginsenosides Rg1, Rg3 and Rb1, which are marker compounds of red ginseng as comparative examples of the present disclosure.

### [Test Example 5] Cytotoxicity

In order to exclude the possibility that the ginsenoside might affect antioxidant effect through cytotoxic activity, cell growth in the presence of the novel ginsenoside GS #10 according to an exemplary embodiment of the present disclosure was evaluated as follows using CCK (Cell Counting Kit)-8.

After adding 10 μL of CCK-8 reagent to SH-SY5Y cells (Dojindo, Md., USA) on a 96-well plate and maintaining at 37° C. for 2 hours, absorbance was measured at 450 nm. Cell viability was represented as the percentage (%) of the absolute optical density of each sample with respect to an untreated sample. The concentrations of the novel ginsenoside GS #10 according to an exemplary embodiment of the present disclosure in the medium for culturing the cells were 0.1, 1, 5, 10, 20 and 50 μM.

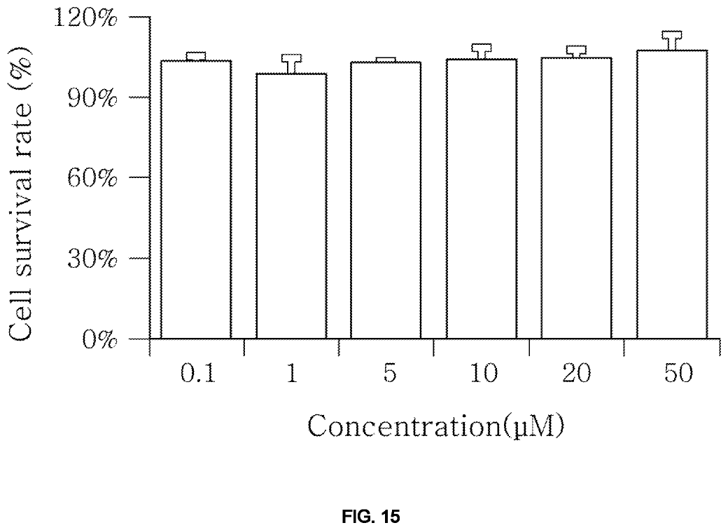

As seen from FIG. 15, the novel ginsenoside GS #10 according to an exemplary embodiment of the present disclosure showed no cytotoxicity up to 50 μM. This result means that the novel ginsenoside according to an exemplary embodiment of the present disclosure can exhibit antioxidant effect without negative effect on cell viability.

The above result suggests that the novel ginsenoside PG-RT₈ according to an exemplary embodiment of the present disclosure has various powerful antioxidant activities and can be used as an antioxidant for pharmaceutical applications.

Hereinafter, formulation examples of the composition according to an exemplary embodiment of the present disclosure are described. However, other various formulations are also possible, and the present disclosure is not limited thereto.

### [Formulation Example 1] Softening Lotion (Skin Lotion)

A softening lotion was prepared by a common method according to the composition described in the following table.

TABLE 3

| Ingredients | Contents (wt %) |
|---|---|
| PG-RT₈ | 0.1 |
| Glycerin | 3.0 |
| Butylene glycol | 2.0 |
| Propylene glycol | 2.0 |
| Carboxyvinyl polymer | 0.1 |
| PEG-12 nonyl phenyl ether | 0.2 |
| Polysorbate 80 | 0.4 |
| Ethanol | 10.0 |
| Triethanolamine | 0.1 |
| Antiseptic, colorant and flavorant | Proper amount |
| Purified water | Balance |

### [Formulation Example 2] Nourishing Lotion (Milk Lotion)

A nourishing lotion was prepared by a common method according to the composition described in the following table.

TABLE 4

| Ingredients | Contents (wt %) |
|---|---|
| PG-RT₈ | 0.1 |
| Glycerin | 3.0 |
| Butylene glycol | 3.0 |
| Propylene glycol | 3.0 |
| Carboxyvinyl polymer | 0.1 |
| Beeswax | 4.0 |
| Polysorbate 60 | 1.5 |
| Caprylic/capric triglyceride | 5.0 |
| Squalane | 5.0 |
| Sorbitan sesquioleate | 1.5 |
| Liquid paraffin | 0.5 |
| Cetearyl alcohol | 1.0 |
| Triethanolamine | 0.2 |
| Antiseptic, colorant and flavorant | Proper amount |
| Purified water | Balance |

### [Formulation Example 3] Massage Cream

A massage cream was prepared by a common method according to the composition described in the following table.

TABLE 5

| Ingredients | Contents (wt %) |
|---|---|
| PG-RT₈ | 0.1 |
| Glycerin | 8.0 |
| Butylene glycol | 4.0 |
| Liquid paraffin | 45.0 |
| β-Glucan | 7.0 |
| Carbomer | 0.1 |
| Caprylic/capric triglyceride | 3.0 |
| Beeswax | 4.0 |
| Cetearyl glucoside | 1.5 |
| Sorbitan sesquioleate | 0.9 |
| Vaseline | 3.0 |
| Paraffin | 1.5 |
| Antiseptic, colorant and flavorant | Proper amount |
| Purified water | Balance |

### [Formulation Example 4] Tablet

After mixing 100 mg of ginsenoside PG-RT₈, 400 mg of lactose, 400 mg of corn starch and 2 mg of magnesium stearate, a tablet was prepared by tableting the mixture according to a common method.

### [Formulation Example 5] Capsule

After mixing 100 mg of ginsenoside PG-RT₈, 400 mg of lactose, 400 mg of corn starch and 2 mg of magnesium stearate, a capsule was prepared by filling the mixture in a gelatin capsule according to a common method.

### [Formulation Example 6] Granule

After mixing 50 mg of ginsenoside PG-RT₈, 250 mg of anhydrous crystalline glucose and 550 mg starch, the mixture was formed into a granule using a fluidized-bed granule and then filled in a pouch.

### [Formulation Example 7] Drink

After mixing 50 mg of ginsenoside PG-RT₈, 10 g of glucose, 0.6 g of citric acid and 25 g of oligosaccharide syrup and adding 300 mL of purified water, 200 mL of the mixture was filled in a bottle. After the bottle was filled, a drink was prepared by sterilizing the content at 130° C. for 4-5 seconds.

### [Formulation Example 8] Caramel Formulation

A caramel was prepared by mixing 50 mg of ginsenoside PG-RT₈, 1.8 g of corn syrup, 0.5 g of skim milk, 0.5 g of soy lecithin, 0.6 g of butter, 0.4 g of hydrogenated vegetable oil, 1.4 g of sugar, 0.58 g of margarine and 20 mg of table salt.

### [Formulation Example 9] Health Food

TABLE 6

| Ingredients | Contents | |
|---|---|---|
| PG-RT₈ | 100 | mg |

Vitamin mixture

| | | |
|---|---|---|
| Vitamin A acetate | 70 | μg |
| Vitamin E | 1.0 | mg |
| Vitamin B₁ | 0.13 | mg |
| Vitamin B₂ | 0.15 | mg |
| Vitamin B₆ | 0.5 | mg |
| Vitamin B₁₂ | 0.2 | μg |
| Vitamin C | 10 | mg |
| Biotin | 10 | μg |
| Nicotinamide | 1.7 | mg |
| Folic acid | 50 | μg |
| Calcium pantothenate | 0.5 | mg |

Mineral mixture

| | | |
|---|---|---|
| Ferrous sulfate | 1.75 | mg |
| Zinc oxide | 0.82 | mg |
| Magnesium carbonate | 25.3 | mg |
| Potassium phosphate monobasic | 15 | mg |
| Calcium phosphate dibasic | 55 | mg |
| Potassium citrate | 90 | mg |
| Calcium carbonate | 100 | mg |
| Magnesium chloride | 24.8 | mg |

Although the above composition of the vitamin and mineral mixtures was presented as an example relatively suitable for health foods, the composition may be varied as desired. According to a common health food preparation method, the above ingredients may be mixed and then prepared into a granule, which may be used to prepare a health food composition according to a common method.

### [Formulation Example 10] Health Drink

TABLE 7

| Ingredients | Contents | |
|---|---|---|
| PG-RT₈ | 10 | mg |
| Citric acid | 1000 | mg |
| Oligosaccharide | 100 | g |
| Plum concentrate | 2 | g |
| Taurine | 1 | g |
| Purified water | Balance | |
| Total volume | 900 | mL |

As shown in the above table, a balance of purified water was added to make a total volume of 900 mL, and the above ingredients were mixed according to a common method for preparing a healthy drink, and heated at 85° C. under stirring for about 1 hour. Then, the resulting solution was filtered and collected in a sterilized 2-L container, sterilized, sealed, and then stored in a refrigerator for use in preparation of a healthy drink composition.

### [Formulation Example 11] Injection

An injection was prepared according to a common method with the composition described in the following table.

TABLE 8

| Ingredients | Contents |
|---|---|
| PG-RT₈ | 10-50 mg |
| Sterile distilled | Proper amount |
| water for injection | |
| pH control agent | Proper amount |

The present disclosure may provide the following exemplary embodiments.

A first exemplary embodiment may provide an antioxidant composition comprising

(20S,24R)-6-O-β-D-glucopyranosyl(1->2)-β-D-glucopyranoside-dammar-3-one-20,24-epoxy-6a,12b,25-triol, a pharmaceutically acceptable salt thereof, a hydrate thereof or a solvate thereof as an active ingredient.

A second exemplary embodiment may provide the composition according to the first exemplary embodiment, wherein the active ingredient has a structure of Chemical Formula 1.

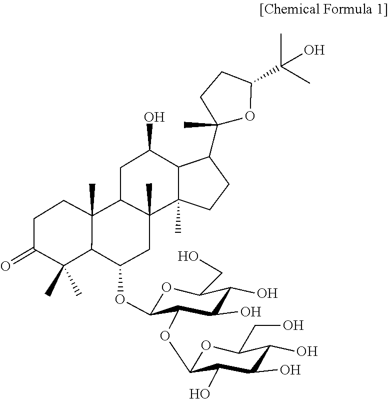

A third exemplary embodiment may provide the composition according to the first exemplary embodiment or the second exemplary embodiment, wherein the active ingredient is one extracted from ginseng seed.

A fourth exemplary embodiment may provide the composition according to any of the first to third exemplary embodiments, wherein the active ingredient inhibits the activity of 2,2-diphenyl-1-picrylhydrazyl (DPPH).

A fifth exemplary embodiment may provide the composition according to any of the first to fourth exemplary embodiments, wherein the active ingredient removes reactive oxygen species (ROS).

A sixth exemplary embodiment may provide the composition according to any of the first to fifth exemplary embodiments, wherein the active ingredient is comprised in an amount of 0.0001-99.9 wt % based on the total weight of the composition.

A seventh exemplary embodiment may provide the composition according to any of the first to sixth exemplary embodiments, wherein the composition is a composition for external application to skin.

An eighth exemplary embodiment may provide the composition according to any of the first to seventh exemplary embodiments, wherein the composition is a cosmetic composition.

A ninth exemplary embodiment may provide the composition according to any of the first to eighth exemplary embodiment, wherein the composition is a food composition.

The above exemplary embodiments are provided for illustration of the present disclosure, but the scope of the present disclosure is not limited thereby. Accordingly, various modifications, changes and substitutions may be made by those of ordinary skill in the art without departing from the meaning and scope of the present disclosure.

1. A method for antioxidation comprising administering a subject in need thereof an effective amount of (20S,24R)-6-O-β-D-glucopyranosyl(1->2)-β-D-glucopyranoside-dammar-3-one-20,24-epoxy-6a,12b,25-triol, a pharmaceutically acceptable salt thereof, a hydrate thereof or a solvate thereof.

2. The method according to claim 1, wherein the (20S,24R)-6-O-β-D-glucopyranosyl(1->2)-β-D-glucopyranoside-dammar-3-one-20,24-epoxy-6a,12b,25-triol has a structure of Chemical Formula 1:

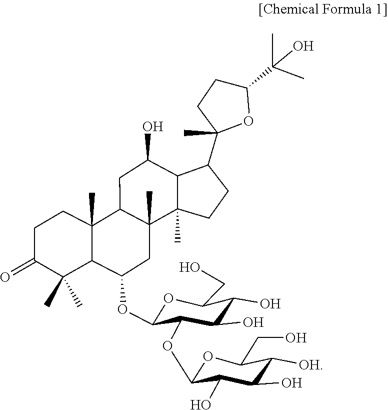

3. The method according to claim 1, wherein the (20S,24R)-6-O-β-D-glucopyranosyl(1->2)-β-D-glucopyranoside-dammar-3-one-20,24-epoxy-6a,12b,25-triol is one extracted from ginseng seed.

4. The composition according to claim 1, wherein the (20S,24R)-6-O-β-D-glucopyranosyl(1->2)-β-D-glucopyranoside-dammar-3-one-20,24-epoxy-6a,12b,25-triol, a pharmaceutically acceptable salt thereof, a hydrate thereof or a solvate thereof inhibits the activity of 2,2-diphenyl-1-picrylhydrazyl (DPPH).

5. The composition according to claim 1, wherein the (20S,24R)-6-O-β-D-glucopyranosyl(1->2)-β-D-glucopyranoside-dammar-3-one-20,24-epoxy-6a,12b,25-triol, a pharmaceutically acceptable salt thereof, a hydrate thereof or a solvate thereof removes reactive oxygen species (ROS).

6. The method according to claim 1, wherein the active ingredient is comprised in a composition,
wherein the active ingredient is comprised in an amount of 0.0001-99.9 wt % based on the total weight of the composition.

7. The method according to claim 1, wherein the active ingredient is comprised in a composition,
wherein the composition is a composition for external application to skin.

8. The method according to claim 1, wherein the active ingredient is comprised in a composition,
wherein the composition is a cosmetic composition.

9. The method according to claim 1, wherein the active ingredient is comprised in a composition,
wherein the composition is a food composition.
